(19) United States
(12) Patent Application Publication
(10) Pub. No.: US 2015/0087039 A1
(43) Pub. Date: Mar. 26, 2015

(54) METHODS FOR ENHANCING THE RECOVERY OF OIL DURING BIOFUEL PRODUCTION

(71) Applicant: ABENGOA BIOENERGY NEW TECHNOLOGIES, LLC, Chesterfield, MO (US)

(72) Inventor: Thomas Carsten Tandy, York, NE (US)

(73) Assignee: ABENGOA BIOENERGY NEW TECHNOLOGIES, LLC, Chesterfield, MO (US)

(21) Appl. No.: 14/382,983

(22) PCT Filed: Mar. 5, 2013
(86) PCT No.: PCT/US2013/029066

§ 371 Date: Sep. 4, 2014

Related U.S. Application Data
(60) Provisional application No. 61/607,684, filed on Mar. 7, 2012.

(51) Int. Cl. C11B 3/00; C11B 3/10; C12P 7/14

(52) U.S. Cl. CPC C11B 3/001; C12P 7/14; C11B 3/003; C11B 3/10; C11B 3/006
USPC 435/162; 435/262; 554/177

(57) ABSTRACT

Methods for producing biofuels that involve enhanced separation of oil in co-product process streams are disclosed. In some embodiments, separation is enhanced by addition of an additive such as a surfactant, yeast and/or salt to the co-product process stream.

### CROSS-REFERENCE TO RELATED APPLICATION

This application claims the benefit of U.S. Provisional Application No. 61/607,684 filed Mar. 7, 2012, which is incorporated herein by reference in its entirety.

### FIELD OF THE DISCLOSURE

The present disclosure relates to production of biofuels and, in particular, to production processes that involve improved separation of oil in co-product process streams. In some embodiments, separation is enhanced by addition of an additive such as a surfactant, yeast and/or salt to the co-product process stream.

### BACKGROUND

Conventional biofuel production processes involve pre-treatment of a feedstock to produce simple sugars which may be fermented to produce the biofuel. Such processes typically involve production of one or more co-products such as various lipids (i.e., oil), fiber and/or protein. Such co-products may be processed prior to or after fermentation to separate and/or purify the co-product into a value-added commercial product. An exemplary process includes separation of oil from dissolved solids and other components in an intermediary process stream such as thin stillage that is produced during ethanol fermentation. Oil has a relatively higher commercial value per unit weight which may justify its separation from such intermediary process streams. Such separation may be performed by one or more processes that take advantage of differences in specific gravity between oil and the other components such as by centrifugation, settling tanks, decanters or horizontal tricanters. Other methods of separation may be used to recover oil such as filtration or solvent extraction.

Conventional oil separation methods have been plagued by poor oil recovery which make such separation techniques less economical. As a result, depending on the economics of the particular biofuel facility, installation of oil-separation equipment may not be economically justified. As a result, many biofuel processing plants do not include such equipment and the oil remains in co-product streams that are often sold as an agricultural feed at a unit weight price much less than oil.

A continuing need exists for economical methods for enhancing the recovery of oil from process streams of biofuel production facilities such as from thin stillage produced as an intermediary stream in corn-ethanol production.

### SUMMARY

One aspect of the present disclosure is directed to a process for recovering oil from thin stillage obtained in the production of a fuel from a carbon source. The thin stillage is an emulsion of water, dissolved solids and oil. Thin stillage is contacted with yeast to enhance separation of oil from the thin stillage. Oil is separated from the yeast-containing thin stillage.

Another aspect of the present disclosure is directed to a process for recovering oil from thin stillage obtained in the production of a fuel from a carbon source. The thin stillage is an emulsion of water, dissolved solids and oil. Thin stillage is contacted with a salt suitable to enhance separation of oil from the thin stillage. Oil is separated from the salt-containing thin stillage.

A further aspect of the present disclosure is directed to a process for producing ethanol from a carbon source containing polysaccharides. The polysaccharides in the carbon source are reduced to produce simple sugars. The simple sugars are fermented to produce ethanol and whole stillage. The whole stillage is separated into a distillers grain component and a thin stillage component. The thin stillage component is contacted with yeast. Oil is separated from the yeast-containing thin stillage component.

Yet another aspect of the present disclosure is directed to a process for producing ethanol from a carbon source containing polysaccharides. The polysaccharides in the carbon source are reduced to produce simple sugars. The simple sugars are fermented to produce ethanol and whole stillage. The whole stillage is separated into a distillers grain component and a thin stillage component. The thin stillage component is contacted with salt. Oil is separated from the salt-containing thin stillage component.

In another aspect of the present disclosure, a method for enhancing the separation of oil from a oil-in-water emulsion involves contacting the emulsion with a yeast. The emulsion includes at least about 1 wt % oil on a dry basis and at least about 5 wt % solids.

In a further aspect of the present disclosure, a method for enhancing the separation of oil from a oil-in-water emulsion involves contacting the emulsion with a salt. The emulsion includes at least about 1 wt % oil on a dry basis and at least about 5 wt % solids.

Various refinements exist of the features noted in relation to the above-mentioned aspects of the present disclosure. Further features may also be incorporated in the above-mentioned aspects of the present disclosure as well. These refinements and additional features may exist individually or in any combination. For instance, various features discussed below in relation to any of the illustrated embodiments of the present disclosure may be incorporated into any of the above-described aspects of the present disclosure, alone or in any combination.

### BRIEF DESCRIPTION OF THE DRAWINGS

FIG. 1 is a block diagram showing exemplary and conventional processing steps used to produce a biofuel from a feedstock; and

FIG. 2 is a block diagram showing exemplary processing steps used to produce a biofuel from a feedstock in accordance with the present disclosure.

Corresponding reference characters indicate corresponding parts throughout the drawings.

### DETAILED DESCRIPTION

In accordance with the present disclosure, one or more additives are added to a process stream used in biofuel production to promote separation of oil from the process stream. Suitable additives that may promote separation of oil include a surfactant, yeast and salt. It is believed that the additive acts to change the physical and/or chemical properties of the process stream in a manner that allows oil to be more readily removed by gravity-based separation processes. It should be noted however that the present disclosure should not be limited to a particular mode of action or additive effect.

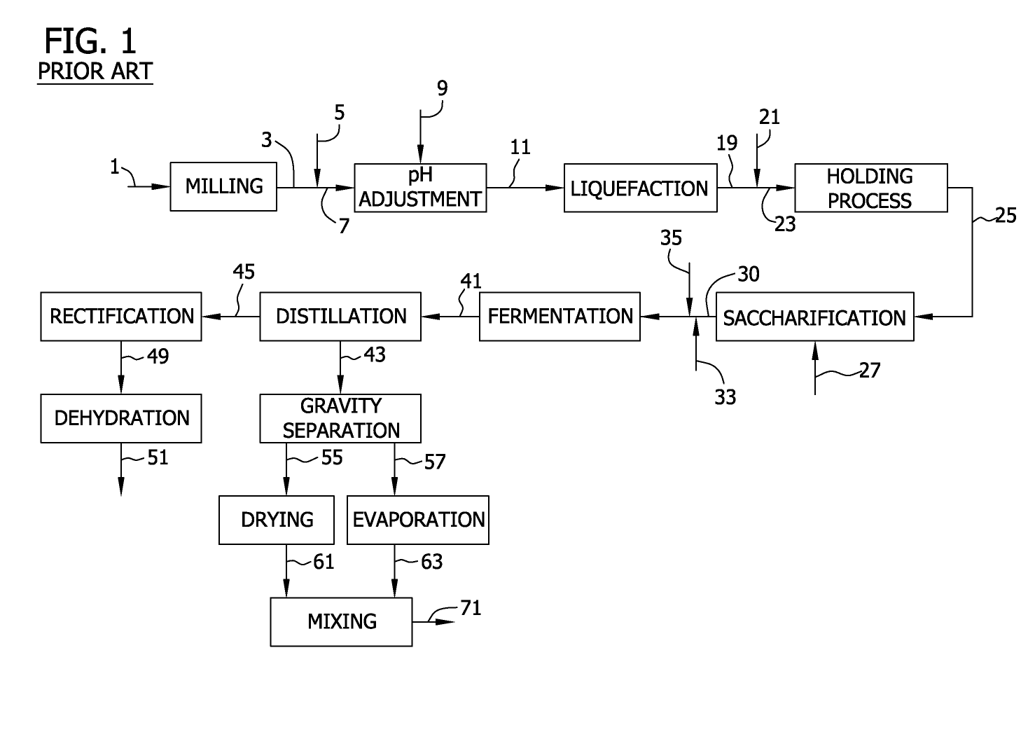

An exemplary process for production of a biofuel is shown in FIG. 1. Any suitable feedstock 1 that contains a polysaccharide that may be reduced into a simple sugar may be used without departing from the scope of the present disclosure. Suitable feedstocks may include cellulose and/or hemicellulose sources and feedstocks that contain starch. In addition to reducible polysaccharides, the feedstock also contains an amount of oil. Suitable feedstocks include the seeds and/or fruit components of plants which often contain a large proportion of readily-reducible starch and may also contain oil. Suitable feedstocks include cereal grains such as corn kernels, wheat berries (with or without the hull), oat groats (with or without the hull), as well as the grains of rye, barley, canola, sorghum, rice and quinoa. Various grasses and/or legumes that contain amounts of oil may also be used such as soybeans, clover, switchgrass, bamboo, marram grass, meadow grass, reed, ryegrass, sugar cane, and grasses from the Miscanthus genus. Tubers such as potatoes, cassava, sweet potato, sugar beets and yam may also be used. Material typically considered to be waste products such as corn stover or orange peels may also be used. It should be noted that the recited feedstocks are exemplary and any feedstock that contains reducible polysaccharides and oil (e.g., at least about 0.5 wt % or at least about 2 wt % oil) may be used as well as combinations of the various feedstocks.

In some embodiments, the feedstock is corn kernels (which may be referred to herein simply as “corn”). Corn has a relatively high percentage of starch (e.g., up to about 80 wt %) and is a feedstock commonly used to produce ethanol by fermentation. Corn also includes a relatively large amount of fiber (about 9%) and protein (about 9%) and also includes oil (about 4 wt %).

Any method available to those of skill in the art to produce a biofuel from a feedstock may be used in accordance with the present disclosure. An exemplary process for producing a biofuel from a feedstock is shown in FIG. 1. As shown in FIG. 1 and in accordance with certain embodiments of the present disclosure, the feedstock 1 (e.g., corn kernels) is milled to reduce the particle size of the material and to expose all portion of the feedstock to the operating process. Milling may be performed by, for example, using a hammer mill and screens, to produce a “meal” that is processed without separating out the various component parts of the grain. In some embodiments (e.g., when corn is the feedstock), the grain is dry milled into a meal having an average particle size ranging from about 250 micrometers to about 1200 micrometers, preferably ranging from about 500 micrometers to 750 micrometers. As stated above, after dry milling, the flour comprises all of the grain components, including the protein, starch, fibers, and oil.

The milled material 3 may be combined with one or more aqueous streams 5 including water or recycled streams such as whole or thin stillage or condensates thereof to form a mash. An amount of acid 9 (e.g., sulfuric acid) may be added to the mash to adjust the pH to a pH that facilitates breakdown of the polysaccharide (e.g., starch) in downstream processing and/or to directly cause partial hydrolysis of the polysaccharide. Acid hydrolysis breaks down at least a portion of the starch, cellulose, and/or hemicellulose into smaller fragments. Acid hydrolysis also separates lignin from lignin-cellulose and lignin hemicellulose complexes.

The pH adjusted mash 11 may then be subjected to a liquefaction process which rapidly raises the temperature of the mash and subjects the mash to mechanical shear force. The liquefaction process typically involves direct injection of steam (e.g., as with a steam jet) which breaks up the starch crystals and hydrates the starch by gelatinization thereby promoting acid hydrolysis and improving downstream processing.

The liquefied mash 19 is then combined with an enzyme 21, typically α-amylase, to form a treated mash 23 which may be introduced onto one or more holding tanks to continue the liquefaction process. The α-amylase enzyme catalyzes enzymatic hydrolysis of at least a portion of the starch to form simple sugars, such as glucose, maltose, maltotriose and the like. In some embodiments, the enzyme is added prior to steam injection; however the enzyme is preferably added after steam injection to preserve the enzymes activity.

After liquefaction is complete, the liquefaction product 25 is subjected to one or more saccharification steps in which glucoamylase 27 is added at a reduced temperature relative to liquefaction to reduce the starch components to glucose and other simple sugars such as maltose and maltotriose. Optionally additional enzymes may be used such as protease, cellulase, xylanase, or hemicellulase to catalyze the enzymatic hydrolysis of xylan, cellulose, and hemicellulose to produce simple sugars for fermentation. Optionally, additives such as yeast and/or various nutrients may be added to a portion of the saccharification process stream so as to form a yeast propagation mixture that propagates yeast for use during fermentation.

After saccharification, the enzymatic hydrolysate 30 may be cooled and yeast 33 and additional nutrients 35 are added to the enzymatic hydrolysate to form a primary fermentation mixture. The action of the yeast converts simple C6 sugars (i.e., glucose) into carbon dioxide and ethanol. Alternatively or in addition, yeast (e.g., certain strains of yeast and/or GMO yeast) may be used to convert C5 sugars (e.g., xylose) that are produced to carbon dioxide and ethanol.

The fermented mixture 41 is distilled to separate the ethanol from the mixture. The distillation separates the ethanol into a high wines stream 45 that may be rectified. The rectified stream 49 may then be dehydrated, yielding ethanol 51 suitable for use as fuel or for consumption. The remaining portion of the fermentation mixture after distillation is referred to as “whole stillage” 43. The whole stillage 43 typically comprises protein, cellulose, hemicellulose, fibers, oil, fat, and lignin.

The whole stillage 43 may be separated into a wet distillers grains (“WDG”) portion 55 that includes the insoluble portions of the whole stillage 43 and a thin stillage portion 57 (referred to herein simply as “thin stillage”) by a gravity separation process such as centrifugation or any other suitable method available to those of skill in the art. In accordance with conventional co-product processing, the wet distillers grains 55 are dried yielding dried distillers grains (“DDG”) 61. The thin stillage 57 may be evaporated, yielding condensed distillers solubles (“CDS”) 63. The dried distillers grains and condensed distillers solubles may be combined, yielding dried distillers grains with solubles (“DDGS”) 71. DDGS 71 is commonly used as a feed ration for cattle or other animals.

It should be noted that the above described process and the process illustrated in FIG. 1 for producing ethanol is only exemplary and commercial process not shown or described (e.g., further recycle streams and/or other processing operations) may be included without limitation. The process may be modified and/or alternative production processes may be used without departing from the scope of the present disclosure. The particular processes and operations chosen for use should be highly individualized depending on plant capacity, co-product markets, equipment used and the like. It should be further noted that each operation of the process may be carried out in a single unit or multiple units configured in series or parallel may be used. The various operations may be operated in a continuous manner in which feeds and products are continually introduced and withdrawn or may be operated in a batch or even a semi-batch mode. The exemplified process described above should not be considered in a limiting sense.

It should also be noted that while the process of the present disclosure has been described in the context of a dry mill biofuel plant, wet mill processes may also be used without departing from the scope of the present disclosure. In wet milling, the feedstock is first soaked or steeped in an aqueous solution such as a sulfurous solution to soften the material. Wet grinding is used to release the oil-containing germ and coarse fiber from the endosperm. In typical wet mill applications, the fiber and germ are separated and the endosperm further processed and separated into starch and protein fractions. The separated starch streams from a wet-mill can advantageously serve as a feedstock to the ethanol fermentation process due to the reduced amount of non-fermentable matter entering the process and the ability to capture the oil, protein, and fiber separately which have economic value for human food and other applications (see e.g., U.S. Pat. No. 3,236,740 which is incorporated herein for all relevant and consistent purposes). A portion of the aqueous stream used to steep the feedstock may be condensed to concentrate the soluble material therein. The oil recovery process of the present disclosure described below may also be applied to this aqueous stream (referred to in the art as a “light steepwater” stream) or the condensed stream (referred to in the art as a “heavy steepwater” stream) or to an intermediate process stream of the wet mill process that contains oil.

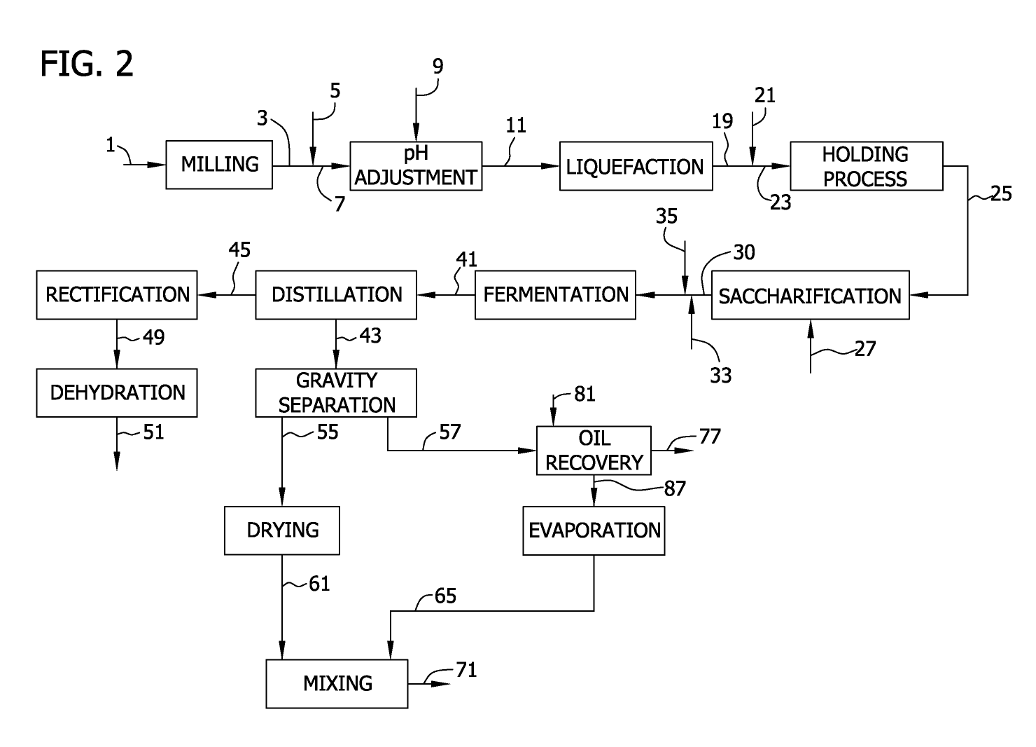

In accordance with the present disclosure and as shown in FIG. 2, the thin stillage 57 may be subjected to an oil recovery process to recover a portion of the oil 77 from the thin stillage. Any method available to those of skill in the art may be used to separate oil 77 from the thin stillage 57. Gravity separation methods have been relatively effective due to the difference in specific gravity between oil and the remaining stillage and due to oil being relatively immiscible in the thin stillage. Suitable methods include centrifugation or use of settling tanks, decanters or horizontal tricanters. Once oil is removed, the oil-depleted thin stillage 87 is concentrated by evaporation and the condensed distillers solubles 65 may be mixed with dried distillers grains 61 to produce DDGS 71.

In accordance with the present disclosure, an additive 81 is added to the thin stillage 57 to promote separation of oil 77 from thin stillage. A number of additives may be used to promote separation of oil including surfactants, salt and/or yeast. Generally the additive is added to the thin stillage 57; however it should be noted that the additive may be added upstream to promote mixing (e.g., prior to gravity separation such as centrifugation). In this regard, in embodiments wherein the treated stream is aqueous (e.g., thin stillage), the stream is an oil-in-water emulsion. The additive is believed to change the physical and/or chemical properties of the emulsion which facilitates better separation of oil from the emulsion.

In this regard, the process stream to which the additive is added need not be a “thin stillage” stream as referred to by those of skill in the biofuel field. Any process stream that contains an amount of oil and other components may be treated with the additive to facilitate separation of oil from the stream. Preferably the stream is aqueous. The stream (whether a thin stillage stream or another process stream) may contain an amount of solids within the stream and in some embodiments contains at last about 5 wt % solids. Such solids may be suspended in the stream or wholly or partially dissolved in the stream and may be measured by evaporating all water from the stream and determining the weight of the remaining solids. In some embodiments, the stream contains at least about 10 wt % solids, at least about 15 wt %, at least about 20 wt %, at least about 30 wt %, at least about 35 wt %, from about 9 wt % to about 42 wt %, from about 15 wt % to about 42 wt % or from about 15 wt % to about 35 wt % solids. The stream may contain at least about 1 wt % oil on a dry basis or at least about 3 wt %, at least about 5 wt %, at least about 10 wt %, from about 1 wt % to about 40 wt %, from about 1 wt % to about 20 wt %, from about 1 wt % to about 15 wt %, from about 3 wt % to about 20 wt % or from about 5 wt % to about 20 wt % oil on a dry basis.

Generally the temperature of the stream to which the oil-separating additive is added need not be heated or cooled prior to or after addition of the additive. However as generally higher temperatures promote separation of oil from stillage, it is preferred that the temperature of the thin stillage be at least about 40° C. after addition of the additive and during oil separation. In other embodiments, the temperature of the additive-containing stream is at least about 50° C., at least about 60° C., at least about 70° C., at least about 80° C., at least about 90° C., from about 40° C. to about 100° C., from about 50° C. to about 100° C., from about 60° C. to about 90° C. or from about 70° C. to about 90° C. after addition of the additive and during oil separation.

In some embodiments, a surfactant is added as an additive to promote separation of oil. Without being bound to any particular theory, it is believed that surfactants stabilize the oil-in-water emulsion (e.g., act as an emulsifier); however surfactants also act to separate the oil from the mixture when a gravitational separation process is applied to the treated stream. Without being bound to any particular theory, it is believed that the surfactant may free oil that is attached to other hydrophobic materials that are nearly as dense as or even denser than the emulsion itself which prevents them from being separated by gravity separation methods. It should be noted that other mechanisms may act to increase separation and the present disclosure should not be limited to a particular mechanism or mode of action.

Generally any surfactant available to those of skill in the art may be used. Preferable surfactants include polysorbates (e.g., POLYSORBATE 80), sorbitan stearates (e.g., sorbitan monostearate), emulsifying waxes, cetearyl alcohol and polyoxyethylene ethers (e.g., CETEARETH compounds such as CETEARETH 20). In some embodiments, the surfactant is chosen from polysorbates and sorbitan stearates (e.g., sorbitan monostearate). The surfactant may be added at a rate of at least about 0.001 wt % (e.g., at least about 0.005 wt %, at least about 0.01 wt %, at least about 0.1 wt %, at least about 1 wt %, from about 0.001 wt % to about 5 wt %, from about 0.01 wt % to about 5 wt % or from about 0.001 wt % to about 1 wt %). Preferably the surfactant is contacted with the stream for a period of time prior to oil separation to allow the surfactant to sufficiently mix into the material and alter the oil-in-water emulsion. In some embodiments, oil is separated after at least about 5 minutes after the treated stream is contacted with surfactant or, as in other embodiments, at least about 15 minutes, at least about 30 minutes, at least about 45 minutes, at least about 1 hour, at least about 3 hours, or at least about 6 hours after the stream is contacted with surfactant (e.g., from about 5 minutes to about 10 hours, from about 5 minutes to about 8 hours, from about 1 hour to about 8 hours, from about 5 minutes to about 1 hour or from about 15 minutes to about 1 hour).

In some embodiments, yeast is added to the process stream (e.g., to the thin stillage stream) to improve separation of oil. Typically the species of yeast that is used is Saccharomyces cerevisiae including the various strains thereof; however other species and types of yeast may be used without limitation. In this regard, the yeast need not be in an active state prior to addition to the stream to achieve the emulsion-altering effect; rather, dry yeast (i.e., inactive) or even dead yeast may be used to achieve the effect. It is believed that most of the improvement of oil separation attributed to yeast does not result from yeast consuming one or more constituents in the emulsion (e.g., does not result from consumption of glycerol when thin stillage is used) but rather the effect is primarily caused by a physical or chemical modification of the emulsion. However, the present disclosure should not be limited to a particular mode of action and the improvement in separation that is observed may be attributed to physical, chemical and/or biological action without limitation.

In various embodiments of the present disclosure, at least about 0.05 g of yeast (on a dry basis of yeast) is added per liter of treated stream (e.g., thin stillage) or, as in other embodiments, at least about 0.5 g, at least about 1 g, at least about 5 g or from about 0.05 g to about 100 g, from about 0.5 g to about 100 g or from about 1 g to about 50 g of yeast (on a dry basis) is added per liter of treated stream (e.g., thin stillage). Preferably oil is separated after at least about 40 minutes after the treated stream (e.g., thin stillage) is contacted with yeast. In some other embodiments, oil is separated after at least about 50 minutes, from about 40 minutes to about 120 minutes, or from about 40 minutes to about 90 minutes after contacting the stream with yeast.

In some embodiments, in addition to yeast, the thin stillage may be contacted with a surfactant as described above. Suitable surfactants include surfactants that are packaged with the yeast in commercial formulations of the yeast. The surfactant acts as a stabilizer during storage, transportation and initial hydration of yeast to maximize the amount of viable yeast available during fermentation. Further, the surfactant additionally facilitates separation of oil from the emulsion.

In some embodiments, alternatively or in addition to yeast and/or a surfactant, salt is added to the treated stream (e.g., thin stillage). Without being bound to any particular theory, it is believed that salt acts to increase the density of the water-in-oil emulsion which makes the oil more buoyant in the emulsion which allows the oil to be better separated in gravity-based separation processes (e.g., centrifugation, tricanters and the like). Further, salt enhances the hydrophobic effect in the emulsion which facilitates separation of oil from the emulsion.

Preferably the salt that is added is suitable for animal consumption as the salt will typically be present in a feed co-product (e.g., DDGS). The salt may be chosen from various chloride salts including CaCl₂, KCl, NaCl and MgCl₂. Preferably the salt is NaCl as NaCl has been found to be effective in enhancing separation of oil from the emulsion. Salt may be added in a solid form or as a brine. Preferably a brine is used as brines may enhance the oil separation effect greater than solid salt addition.

Salt may be added at a rate of at least about 0.005 g per liter of thin stillage. In other embodiments, salt is added at a rate of at least about 0.05 g per liter of thin stillage or at least about 0.5 g, at least about 1 g, at least about 2 g, at least about 5 g, from about 0.005 g to about 100 g, from about 0.05 g to about 50 g, from about 0.05 g to about 10 g or from about 0.5 g to about 2 g of salt is added per liter of thin stillage. It has been found that once mixed into the stream, salt nearly immediately enhances separation of oil. In some embodiments of the present disclosure, oil is separated less than about 1 hour after the treated stream (e.g., thin stillage) is contacted with salt or less than about 30 minutes, less than about 15 minutes, less than about 10 minutes, less than about 1 minute or from about 0 minutes to about 1 hour after the treated stream is contacted with salt.

It should be noted that the additive may be added to the emulsion (e.g., thin stillage) in a continuous manner or may be added batch-wise or semi-batchwise without departing from the scope of the present disclosure.

The amount of oil separated from thin stillage may depend on the starting amount of oil in stillage. As illustrated in the Examples below, rates of recovery of oil when salt or yeast is added may be at least about 1% vol/vol or, as in other embodiments, at least about 1.5% vol/vol, at least about 2% vol/vol, at least about 2.5% vol/vol or even at least about 3% vol/vol (e.g., from about 1% vol/vol to about 10% vol/vol, from about 1% vol/vol to about 6% vol/vol, from about 1.5% vol/vol to about 10% vol/vol or from about 2% vol/vol to about 8% vol/vol).

### EXAMPLES

The processes of the present disclosure are further illustrated by the following Examples. These Examples should not be viewed in a limiting sense.

### Example 1

### Effect of Temperature on Oil Separation when Yeast is Added

Yeast was added to thin stillage prepared as part of ethanol production from corn feedstock. Thin stillage was at a temperature of either 32° C. or 85° C. Thin stillage was maintained at this initial temperature for a period of 1-24 hours (see Example 2). The solids content of the thin stillage that was tested was 35 wt %. In each run, yeast (RED STAR®, Fermentis (Marcq-en-Baroeul, France)) was added in its dry form at a rate of 1 g per 200 ml of thin stillage. The yeast contained an amount of sorbitan monostearate.

After yeast was added and the yeast-containing thin stillage was maintained at its holding temperature for 1-24 hours, thin stillage was introduced into a 50 ml lab centrifuge (5804R, Eppendorf (Hamburg, Germany)) to separate oil from the thin stillage. In some runs the temperature of the yeast was maintained at its initial temperature (32° C. or 85° C.) and in other runs it was heated to 85° C. before oil separation. After centrifuging for 30 minutes at 1000G, the top 15 ml of the tube was transferred to a new tube and placed in a 15 ml lab centrifuge (Centra-CL2, Thermo IEC (Waltham, Mass.)). The sample was centrifuged in the 15 ml centrifuge for 5 minutes at 1000G. The oil fraction in the 15 ml tube was measured and was calculated for the original 50 ml portion. The oil-separating effect of addition of yeast is shown in Table 1 below. Oil yield is reported as a range of observed values.

TABLE 1
Effect of Yeast Addition at Various Holding and Separation Temperatures.

| | | | | Enhanced | |
|---|---|---|---|---|---|
| | Temperature | | | Oil | |
| | of Thin | | | Separation | |
| | Stillage | | | Effect | |
| | During Yeast | Oil | Oil | Relative to | |
| | Addition and | Separation | Separation | Control | Oil Yield |
| Run | Hold Time | Temperature | Observed? | Observed? | (% v/v) |
| Control | 32° C. | 32° C. | No | — | — |
| (no | | | | | |
| yeast) | | | | | |
| Control | 32° C. | 85° C. | Yes | — | 0.3-1.0 |
| (no | | | | | |
| yeast) | | | | | |
| 1 | 32° C. | 32° C. | No | No | — |
| 2 | 32° C. | 85° C. | Yes | Yes | 2.67-6.08 |
| 3 | 85° C. | 85° C. | Yes | Yes | 1.05-3.27 |

As can be seen from Table 1, yeast enhanced separation of oil in all runs in which oil separation was conducted at a temperature above 32° C. No oil separation was achieved at a separation temperature of 32° C. for both the control and when yeast was added.

### Example 2

### Effect of Hold Time on Oil Separation when Yeast is Added

Yeast was added to thin stillage and oil separation was conducted according to the process of Example 1. Oil separation was performed at 85° C. for each run. Hold time was varied over several runs for hold temperatures of 32° C. and 85° C. The results for each run are shown in Table 2 below.

TABLE 2
Effect of Yeast Addition at Various Holding Times.

| | | | Enhanced Oil | |
|---|---|---|---|---|
| | Temperature of Thin | | Separation Effect | |
| Hold | Stillage During | Oil | Relative to | |
| Time | Yeast Addition and | Separation | Control | Oil Yield |
| (hr) | Hold Time | Observed? | Observed? | (% v/v) |
| 0.5 | 32° C. | No | No | — |
| 1 | 32° C. | Yes | Yes | 2.67-4.44 |
| 2 | 32° C. | Yes | Yes | 3.72-5.03 |
| 3 | 32° C. | Yes | Yes | 4.41-5.43 |
| 4 | 32° C. | Yes | Yes | 3.43-5.29 |
| 24 | 32° C. | Yes | Yes | 5.28-6.08 |
| 0.5 | 85° C. | No | No | — |
| 1 | 85° C. | Yes | Yes | 1.05-1.85 |
| 2 | 85° C. | Yes | Yes | 1.72-2.39 |
| 3 | 85° C. | Yes | Yes | 2.17-2.84 |
| 4 | 85° C. | Yes | Yes | 2.01-3.27 |

As can be seen from Table 2, yeast enhanced the oil separation for every run having a hold time above 0.5 hours.

### Example 3

### Effect of Varying the Source/Viability of Yeast on Oil Separation

The process of Example 1 was performed for several sources of yeast: hydrated yeast, dry yeast and propagated yeast. Oil separation was performed at 85° C. for each source. The yeast loading for each source was the same. Comparison of the oil yield (% vol/vol) for each type is shown in Table 3 below.

TABLE 3
Oil Yield for Various Sources of Yeast

| Oil Yield in | | 1 hr | 2 hr | 3 hr | 4 hr | 24 hr |
|---|---|---|---|---|---|---|
| 50 ml Sample | Hold | hold | hold | hold | hold | hold |
| (% vol/vol) | Temperature | time | time | time | time | time |
| Hydrated | 32° C. | 4.44 | 5.03 | 5.43 | 5.29 | 6.08 |
| Dry Yeast | 32° C. | 4.04 | 4.81 | 5.47 | 5.50 | 5.93 |
| York Prop | 32° C. | 2.67 | 3.72 | 4.41 | 3.43 | 5.28 |
| Hydrated | 85° C. | 1.06 | 1.72 | 2.17 | 2.01 | NA |
| Dry Yeast | 85° C. | 1.05 | 2.39 | 2.84 | 3.27 | NA |
| York Prop | 85° C. | 1.85 | 2.29 | 2.65 | 2.95 | NA |

As can be seen from Table 3, use of hydrated and dry yeast resulted in the best oil separation. The oil yields for samples processed with a 32° C. hold temperature were most improved.

### Example 4

### Effect of Varying Oxygen Availability for Yeast on Oil Separation

Hydrated yeast was added to thin stillage and the thin stillage was maintained under aerobic or anaerobic conditions during the hold time. Two hold times were tested: 30 minutes and 4 hours. The hold time was conducted at 32° C. and separation was performed at 85° C. The oil yield is shown in Table 4 below.

TABLE 4
Oil Yield under Aerobic and Anaerobic Conditions

| Condition | Hold Time | Oil Yield (% vol/vol) |
|---|---|---|
| Aerobic | 30 minutes | no oil |
| Anaerobic | 30 minutes | no oil |
| Aerobic | 4 hours | 2.66 |
| Anaerobic | 4 hours | 2.86 |

As can be seen from Table 4, oil yield was similar under aerobic and anaerobic conditions. This supports the proposition that the activity of the yeast may have a relatively small or even no effect on oil separation.

### Example 5

### Effect of Salt Loading Rate on Oil Separation

Salt was added at various loading rates to thin stillage prepared as part of ethanol production from corn feedstock. Thin stillage containing the salt was held at a temperature of 85° C. for a hold time of 0, 0.5, 1, 2, or 3 hours before oil separation. The solids content of the thin stillage that was tested was 35 wt %. Two sources of salt were used: brine salt (26%) and granular salt. The oil yield for 50 ml samples was determined according to the process of Example 1. The averaged results of the separation are shown in Table 5 below.

TABLE 5
Effect of Salt Loading Rate on Oil Separation

| Oil Yield in | Salt | No | 0.5 hr | 1 hr | 2 hr | 3 hr |
|---|---|---|---|---|---|---|
| 50 ml Sample | Concentration | hold | hold | hold | hold | hold |
| (% vol/vol) | (% w/v) | time | time | time | time | time |
| Brine | 0.53 | 4.35 | 5.02 | 5.58 | 4.94 | 6.03 |
| Brine | 1.06 | 4.10 | 4.69 | 5.42 | 5.48 | 6.13 |
| Brine | 2.11 | 2.69 | 3.73 | 4.35 | 3.31 | 5.20 |
| Granular Salt | 0.53 | 1.04 | 1.76 | 2.19 | 2.03 | NA |
| Granular Salt | 1.06 | 1.09 | 2.41 | 2.89 | 3.33 | NA |
| Granular Salt | 2.11 | 1.82 | 2.24 | 2.59 | 2.94 | NA |

A control was run (not shown in Table 5) in which salt was not added. No oil separation was observed for the control. As can be seen from Table 5, oil separation was enhanced at each loading rate for brine and granular salt addition. Salt added as a brine was more effective in separating oil than granular salt.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above apparatus and methods without departing from the scope of the disclosure, it is intended that all matter contained in the above description and shown in the accompanying figures shall be interpreted as illustrative and not in a limiting sense.

1. A process for recovering oil from thin stillage obtained in the production of a fuel from a carbon source, the thin stillage being an emulsion comprising water, dissolved solids and oil, the process comprising:
contacting thin stillage with yeast to enhance separation of oil from the thin stillage; and
separating oil from the yeast-containing thin stillage.

2. The process as set forth in claim 1 wherein the carbon source is a cereal grain selected from the group consisting of corn kernels, wheat berries, oat groats, rye, barley, canola, sorghum, rice and quinoa.

3. The process as set forth in claim 1 wherein the carbon source is corn kernels.

4. (canceled)

5. The process as set forth in claim 1 wherein the thin stillage comprises at least about 5 wt % solids.

6. The process as set forth in claim 1 wherein the thin stillage is maintained at a temperature of at least about 40° C. after contacting thin stillage with yeast and during oil separation.

7. The process as set forth in claim 1 wherein at least about 0.05 g of yeast on a dry basis of yeast is added per liter of thin stillage.

8. (canceled)

9. The process as set forth in claim 1 wherein the yeast does not consume glycerol in the thin stillage after contacting the thin stillage.

10. The process as set forth in claim 1 wherein the yeast chemically modifies the emulsion.

11. (canceled)

12. The process as set forth in claim 1 wherein oil is separated from the yeast-containing thin stillage by a gravity separation process.

13. (canceled)

14. The process as set forth in claim 1 comprising contacting the thin stillage with a surfactant to enhance separation of oil from the thin stillage.

15. (canceled)

16. The process as set forth in claim 1 wherein the amount of oil separated from the yeast-containing thin stillage is at least about 1% vol/vol.

17. A process for recovering oil from thin stillage obtained in the production of a fuel from a carbon source, the thin stillage being an emulsion comprising water, dissolved solids and oil, the process comprising:
contacting thin stillage with a salt suitable to enhance separation of oil from the thin stillage; and
separating oil from the salt-containing thin stillage.

18. The process as set forth in claim 17 wherein the carbon source is a cereal grain selected from the group consisting of corn kernels, wheat berries, oat groats, rye, barley, canola, sorghum, rice and quinoa.

19-22. (canceled)

23. The process as set forth in claim 17 wherein at least about 0.005 g of salt is added per liter of thin stillage.

24-25. (canceled)

26. The process as set forth in claim 17 wherein the salt is a chloride salt.

27. The process as set forth in claim 17 wherein the salt is chosen from the group consisting of CaCl₂, KCl, NaCl, and MgCl₂.

28-32. (canceled)

33. A process for producing ethanol from a carbon source containing polysaccharides, the process comprising:
reducing the polysaccharides in the carbon source to produce simple sugars;
fermenting the simple sugars to produce ethanol and whole stillage;
separating the whole stillage into a distillers grain component and a thin stillage component; and
recovering oil from the thin stillage component according to the process of claim 1.

34-47. (canceled)

48. A process for producing ethanol from a carbon source containing polysaccharides, the process comprising:
reducing the polysaccharides in the carbon source to produce simple sugars;
fermenting the simple sugars to produce ethanol and whole stillage;
separating the whole stillage into a distillers grain component and a thin stillage component;
recovering oil from the thin stillage component according to the process of claim 17.

49-62. (canceled)

63. A method for enhancing the separation of oil from a oil-in-water emulsion, the emulsion comprising at least about 1 wt % oil on a dry basis and at least about 5 wt % solids, the process comprising contacting the emulsion with a yeast or with a salt.

64-78. (canceled)

79. The process as set forth in claim 17 comprising contacting the thin stillage with a surfactant to enhance separation of oil from the thin stillage.
